(19) United States
(12) Patent Application Publication
(10) Pub. No.: US 2026/0237500 A1
(43) Pub. Date: Aug. 13, 2026

(54) SYSTEMS AND METHODS FOR CONSTRAINT-ENFORCED DIAGNOSTIC RESULT DISPOSITION GOVERNANCE WITH LONGITUDINAL PATTERN DETECTION, ARTIFACT PROBABILITY ESTIMATION, AND ESCALATING CLINICAL OVERSIGHT

(71) Applicant: Richard Joseph Mitchell, TAVARES, FL (US)

(72) Inventor: Richard Joseph Mitchell, TAVARES, FL (US)

(21) Appl. No.: 19/639,218

(22) Filed: Apr. 5, 2026

(51) Int. Cl. G16H 40/20; G16H 10/60; G16H 50/20

(52) U.S. Cl. CPC G16H 40/20; G16H 10/60; G16H 50/20

(57) ABSTRACT

Systems and methods for constraint-enforced governance of abnormal diagnostic result disposition. Each result is assigned a disposition tier by deviation magnitude and patient context. A disposition state machine independent of the originating EHR enforces a constraint preventing silent closure, making clinician inaction an insufficient basis for closure. A decision support engine assembles longitudinal history, artifact probability, guideline recommendations, and a consequence summary for delayed diagnosis; pre-drafted records are confirmable through a single interaction. A tiered batch interface separates high-acuity individual review from low-acuity rapid batch confirmation. A longitudinal pattern governor maintains persistent result threads and escalates governance upon pattern detection. A proactive surveillance engine externalizes the physician worry list as a systematic auditable function. Unresolved results advance through a defined escalation hierarchy including patient notification. An append-only audit registry preserves the information state at each decision. Guideline-concordant dispositions require fewer clinician interactions than non-documented dismissal through existing EHR interfaces.

### CROSS-REFERENCE TO RELATED APPLICATIONS

This application is related to the following co-pending United States patent applications the disclosures of which are incorporated herein by reference in their entireties:

Application Ser. No. 19/575,876, titled “AI System for Clinician-Governed Decision Support in Mental Health,” filed Mar. 23, 2026, directed to ethical constraint enforcement, tiered clinician governance, and cryptographic audit infrastructure for clinical AI decision support. The constraint enforcement and audit registry architectural principles of the present invention share lineage with the genuine oversight framework disclosed therein.

Application Ser. No. 19/575,914, titled “Provider-Independent Hierarchical Constraint Enforcement Architecture for Artificial Intelligence Systems,” filed Mar. 23, 2026. The present invention incorporates the provider-independent hierarchical constraint enforcement architectural principles disclosed therein and applies them to the specific domain of diagnostic result disposition governance.

Application Ser. No. 19/575,894, titled “Deterministic Fleet Continual Learning and Adaptive Validation Architecture for Distributed Autonomous Systems,” filed Mar. 23, 2026, directed to lifecycle governance, deterministic serving, adaptive validation evolution, and staged deployment governance for fleet-scale autonomous systems. The longitudinal pattern governor and adaptive governance threshold mechanisms of the present invention apply related pattern-persistence and threshold-escalation concepts in the clinical domain.

### BACKGROUND OF THE INVENTION

### 1. Technical Field

The present invention relates to health information technology and clinical decision governance systems, and more specifically to systems and methods that enforce documented clinical disposition of abnormal diagnostic results, estimate the probability that abnormal results reflect true physiologic findings versus laboratory or instrumentation artifact, detect clinically significant patterns in longitudinal result sequences, and escalate unresolved results through defined governance hierarchies independent of the electronic health record system in which results originate.

### 2. Background Art

Timely and appropriate clinical follow-up of abnormal diagnostic test results—including laboratory values, radiologic and imaging findings, pathology reports, and microbiology results—is a fundamental requirement of quality medical care. The failure to follow up on clinically significant abnormal results constitutes a well-documented category of preventable medical error with substantial consequences for patient safety and healthcare system liability.

Studies published in peer-reviewed clinical literature consistently document abnormal result follow-up failure rates ranging from approximately 7% to 60% depending on the care setting, result type, and the clinical significance threshold applied. Among the result types with documented high follow-up failure rates are: isolated neutropenia and other hematologic abnormalities; pulmonary nodules and other incidental findings identified on cross-sectional imaging; markedly abnormal tumor markers; significantly abnormal renal function values; and critical values in any laboratory discipline. Each of these failure categories represents a missed window for early diagnosis and intervention in conditions with strong evidence that earlier treatment produces better outcomes.

The dominant mechanism of abnormal result follow-up failure is not physician incompetence or willful neglect. It is a governance architecture failure: the absence of any system-level requirement that a reviewing clinician produce an affirmative, documented disposition for each abnormal result. Under current clinical workflow architecture, a clinician who reviews an abnormal result and elects to take no action—whether by attributing the finding to laboratory artifact, patient non-compliance, or clinical insignificance—may close the result encounter without generating any documentation of that decision. The result is recorded as reviewed; the decision is not recorded at all.

Electronic health record (EHR) systems include alert and notification features that generate flags for abnormal results delivered to provider inboxes. These systems share a common architectural limitation: they are designed as attention-direction mechanisms, not disposition-enforcement mechanisms. An EHR alert can be dismissed by a single keystroke or click without requiring the clinician to document the clinical basis for dismissal. Published research on EHR alert fatigue documents that physicians in high-volume clinical settings override the majority of alerts—in some studies, more than 90%—without meaningfully engaging with the clinical content of the alert. The alert system performs its function (directing clinician attention) but does not enforce the governance outcome that is clinically required (documented and appropriate disposition).

A further limitation of existing clinical decision support and result management systems is their failure to address the temporal fragmentation of the patient's clinical record. A patient seen by a primary care physician, a specialist, and an urgent care provider within a twelve-month period may generate abnormal results at each encounter that are individually below the threshold of urgent action but that, considered as a sequence, indicate a pattern warranting investigation. No existing commercially deployed system aggregates these individual result events into a governed longitudinal pattern and applies escalating disposition requirements based on pattern strength. Each encounter is managed in isolation, and the pattern is never assembled at the point of clinical decision.

A third limitation of existing systems is the absence of artifact probability estimation integrated at the point of result review. Clinicians routinely and legitimately attribute mildly abnormal results to laboratory artifact, specimen handling problems, or pre-analytical variables—a clinically appropriate judgment in many cases. However, this judgment is typically made without quantitative information about the probability that the specific abnormal value, at its specific magnitude, for the specific patient, reflects a true physiologic finding versus a non-physiologic artifact. The result is that artifact attribution is applied inconsistently and sometimes inappropriately, particularly for result categories in which the visual magnitude of the abnormality is not well correlated with the probability of a true physiologic origin.

Prior art systems for assessing signal quality or measurement reliability in physiologic monitoring—including signal quality indices used in fetal heart rate monitoring, pulse oximetry, and electroencephalography—address a technically distinct problem from the artifact probability estimation of the present invention. Signal quality indices in continuous physiologic monitoring systems assess the fidelity of a waveform acquisition channel in real time, flagging degradation attributable to motion artifact, electrode contact failure, or signal noise in continuously sampled physiologic data streams. These indices are properties of the measurement channel, not of the analytical result, and do not involve Bayesian estimation against population-level reference datasets, patient-specific clinical context, or integration with clinical governance state machines. The artifact probability estimation of the present invention operates on discrete analytical results—laboratory values, pathology findings, imaging measurements—and computes a posterior probability that a specific result value reflects a non-physiologic artifact rather than a true finding, based on deviation magnitude, population-level reference data, and patient-specific modifying factors. This estimation is integrated with the governance state machine as a component of the clinician disposition interface, serving a clinical governance function entirely absent from signal quality index systems.

A need therefore exists for systems and methods that: (a) enforce documented clinical disposition of abnormal diagnostic results through architectural constraints that cannot be bypassed by clinician inaction; (b) provide quantitative artifact probability estimates at the point of result review to support evidence-based disposition decisions; (c) maintain longitudinal result sequences for individual patients across care episodes and apply escalating governance requirements based on detected pattern strength; (d) operate independently of any specific EHR system and maintain constraint authority across EHR changes, provider changes, and organizational transitions; and (e) generate patient-safety-grade audit records that preserve the information state at the time of each clinical disposition decision.

### SUMMARY OF THE INVENTION

The present invention provides systems and methods for constraint-enforced diagnostic result disposition governance, hereinafter referred to as the Closed-Loop Diagnostic Oversight Architecture (CLDOA). The invention addresses the foregoing needs through four integrated architectural subsystems: a Disposition Constraint Enforcer (DCE), a Contextual Decision Support Engine (CDSE), a Longitudinal Pattern Governor (LPG), and an Audit and Accountability Registry (AAR).

In one aspect, the invention provides a system for enforcing documented clinical disposition of abnormal diagnostic results comprising: a result ingestion layer that receives result data from one or more clinical information systems via standardized health information exchange interfaces; a result classification engine that assigns a disposition tier to each received result based on result type, value magnitude relative to reference range, patient-specific contextual factors, and configurable clinical governance rules; a disposition state machine that maintains an open, pending, or closed state for each classified result independently of the originating clinical information system; a constraint enforcement module that prevents a result from transitioning to a closed state absent receipt of a disposition record satisfying the documentation requirements associated with the assigned disposition tier; and an escalation engine that advances result governance to a higher authority level when disposition requirements are not satisfied within a defined time window.

In another aspect, the invention provides a method for generating artifact probability estimates for abnormal diagnostic results at the point of clinical review, comprising: accessing a longitudinal result history for the same test type from the patient record; computing a patient-specific artifact probability as a function of the magnitude of deviation from the applicable reference range, the statistical distribution of artifact rates at that deviation magnitude for the test type as derived from a calibrated reference dataset, patient-specific pre-analytical factors including specimen collection flags and medication list, and temporal proximity to prior results of similar magnitude; and presenting the artifact probability estimate to the reviewing clinician concurrently with the disposition requirement interface.

In another aspect, the invention provides a system for governing clinical follow-up of abnormal diagnostic results across longitudinal care episodes comprising: a longitudinal result thread that aggregates result events for individual patients across multiple clinical encounters, provider relationships, and care settings; a pattern detection engine that identifies clinically significant patterns in the aggregated result sequence based on configurable rules governing result frequency, value trend, magnitude persistence, and temporal spacing; a governance escalation module that automatically advances the disposition tier requirement for individual results based on the strength of detected patterns in the patient's longitudinal result thread; and a persistent accountability registry that preserves the complete sequence of disposition decisions, escalation events, and information states with timestamps and clinician attribution.

In another aspect, the invention provides a system for ensuring patient engagement in the governance of unresolved abnormal diagnostic results comprising: a patient notification engine that generates timely plain-language communications to patients regarding abnormal results that have not achieved documented disposition within defined governance windows; a patient-accessible disposition status interface through which patients may view the current governance state of their results and submit follow-up inquiries; and a patient advocacy escalation pathway that routes unresolved results to a designated patient advocate or care coordinator when clinician disposition requirements remain unmet beyond defined escalation thresholds.

The invention may be embodied as a standalone health information system operating independently of any particular EHR platform, as a set of application programming interfaces (APIs) configured to integrate with existing EHR systems via standardized health information exchange protocols including HL7 FHIR, as a software-as-a-service platform accessed by healthcare organizations through a network interface, or as a combination thereof.

The foregoing summary is illustrative only. The scope of the invention is defined by the claims appended hereto.

### BRIEF DESCRIPTION OF THE DRAWINGS

FIG. 1 is a high-level architectural block diagram of the Closed-Loop Diagnostic Oversight Architecture (CLDOA) showing the four primary subsystems and their relationships to external clinical information systems, clinician interfaces, and patient-facing interfaces.

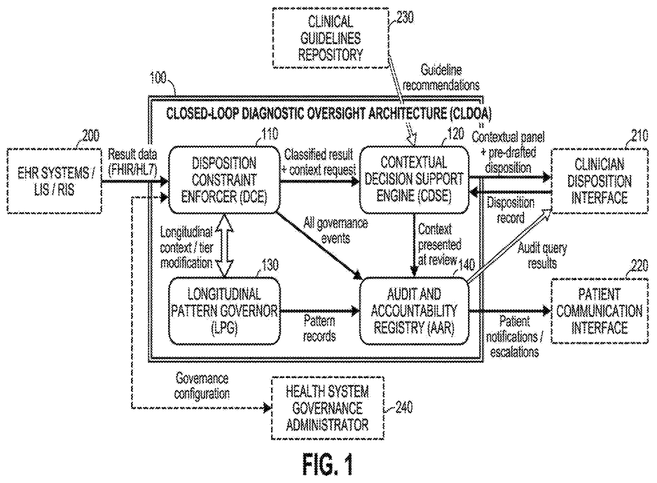

FIG. 2 is a data flow diagram illustrating the result ingestion, classification, and disposition state machine processes from result receipt through disposition closure or escalation.

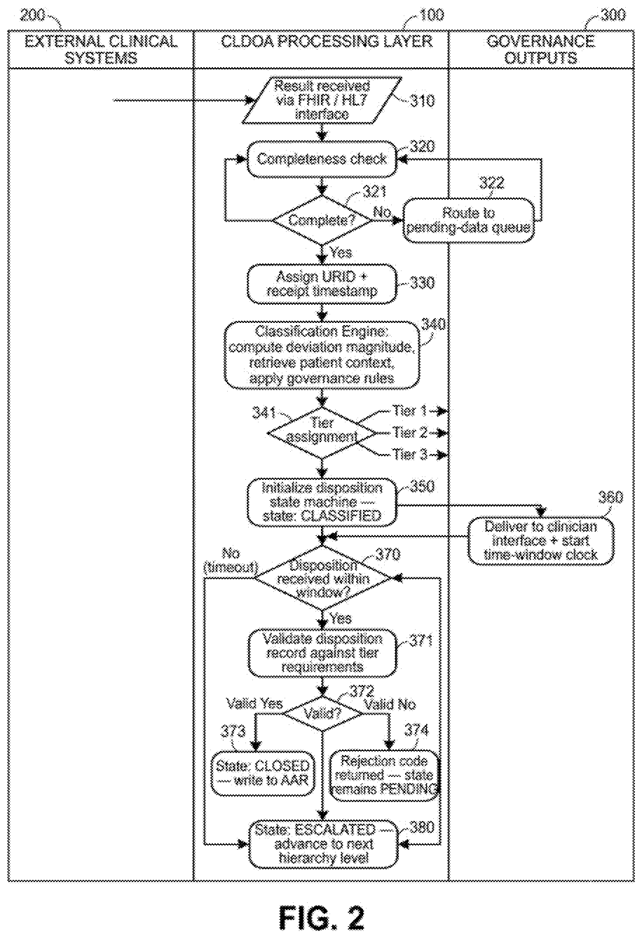

FIG. 3 is a state diagram of the disposition state machine showing all states (Received, Classified, Pending Disposition, Disposition Recorded, Closed, Escalated) and all valid state transitions with their triggering conditions.

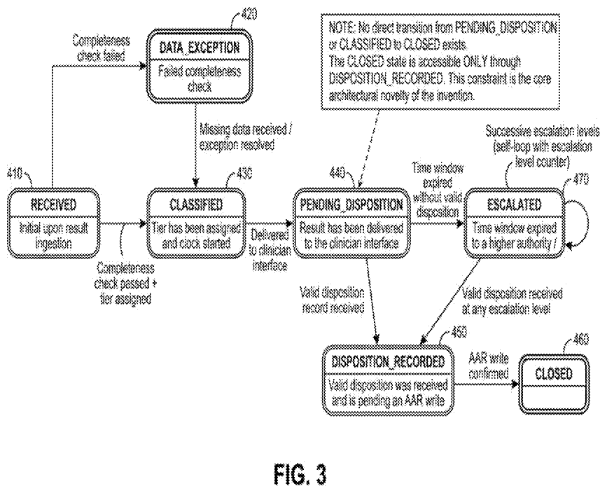

FIG. 4 is a process diagram illustrating the artifact probability estimation method, showing the inputs (reference range deviation magnitude, patient longitudinal history, medication list, specimen handling flags), the computational pathway, and the output integration into the clinician disposition interface.

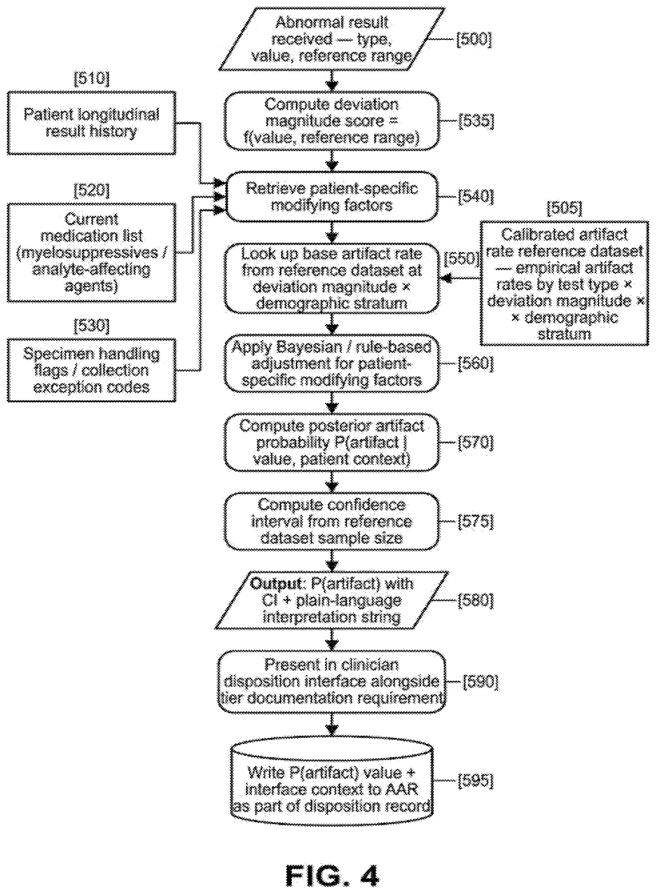

FIG. 5 is a diagram of the longitudinal pattern governor showing the result aggregation process, the pattern detection rule engine, and the feedback pathway by which detected patterns modify disposition tier assignments for current results.

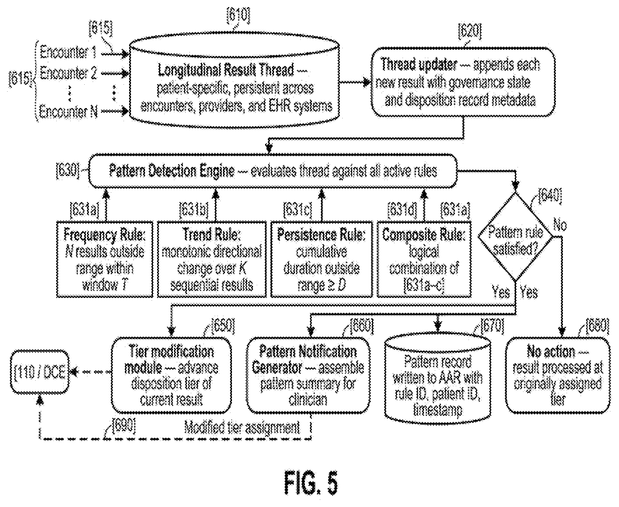

FIG. 6 is a diagram of the escalation hierarchy showing the configured escalation pathway from initial result delivery through attending physician notification, quality officer notification, patient notification, and patient advocate routing.

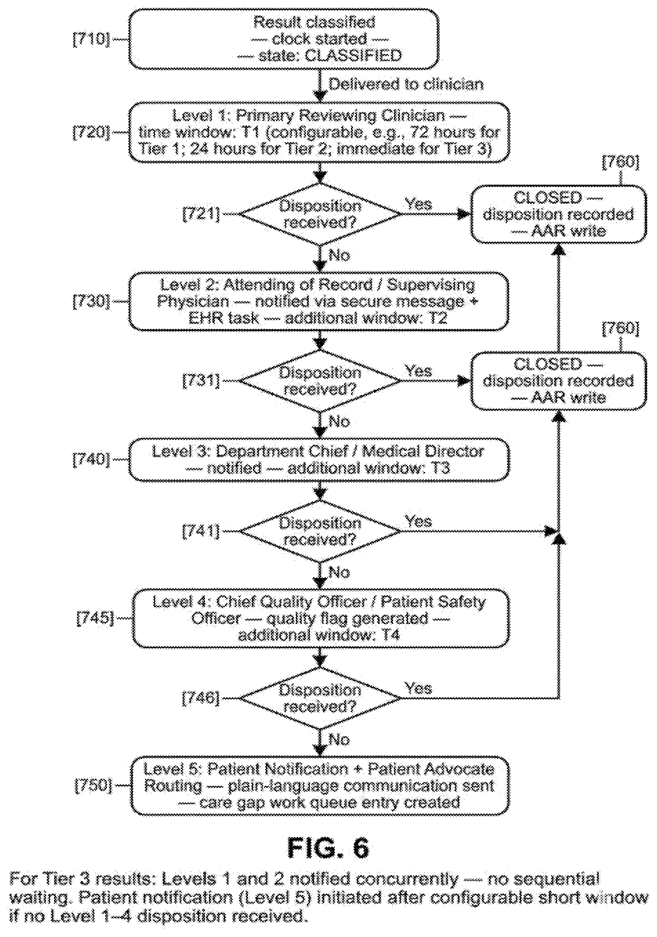

FIG. 7 is a diagram of the Audit and Accountability Registry showing the data elements captured at each disposition event and the query interfaces available for malpractice defense, quality reporting, and regulatory compliance purposes.

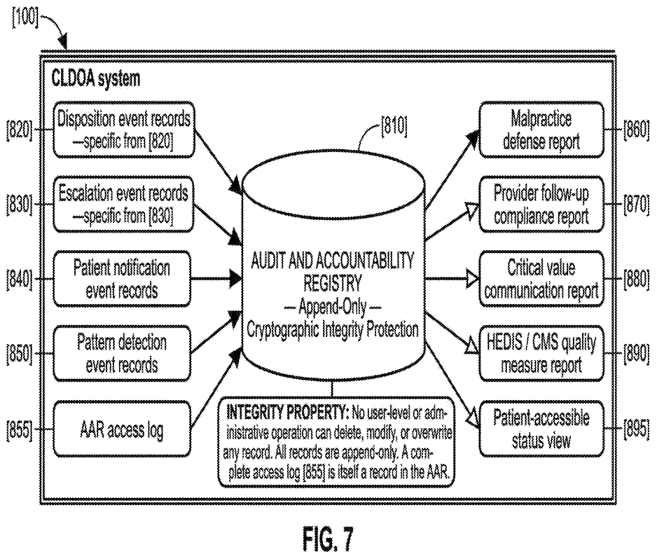

FIG. 8 is an illustrative example of the clinician disposition interface for a case of isolated neutropenia, showing the contextual decision support panel, the artifact probability estimate, the disposition requirement for the assigned tier, and the available disposition options.

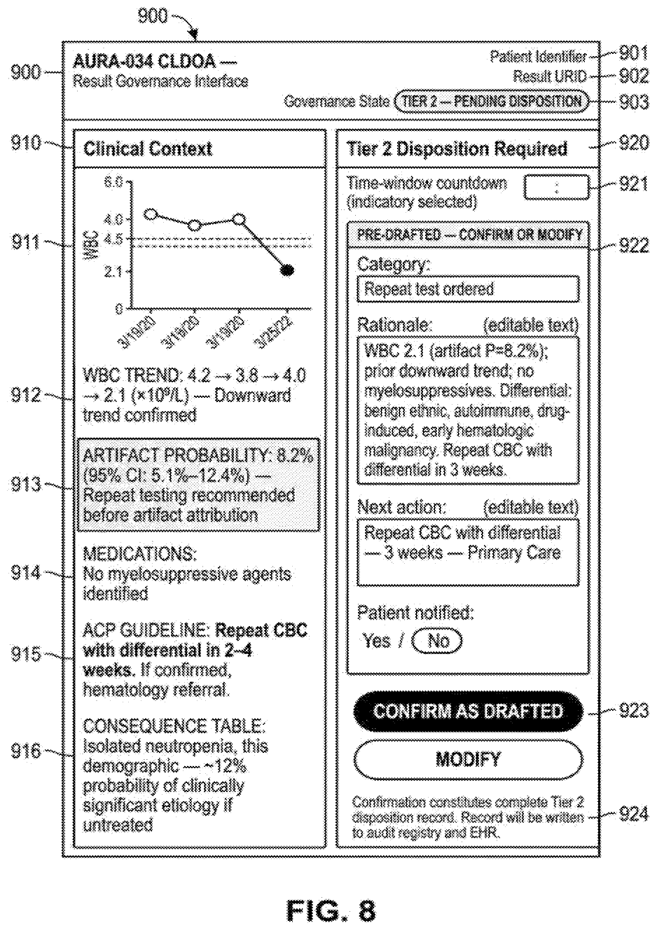

FIG. 9 is a system integration diagram showing the CLDOA operating as an independent governance layer receiving inputs from multiple EHR systems via HL7 FHIR APIs and providing governance outputs through multiple channels including EHR write-back, secure messaging, patient portal, and quality reporting interfaces.

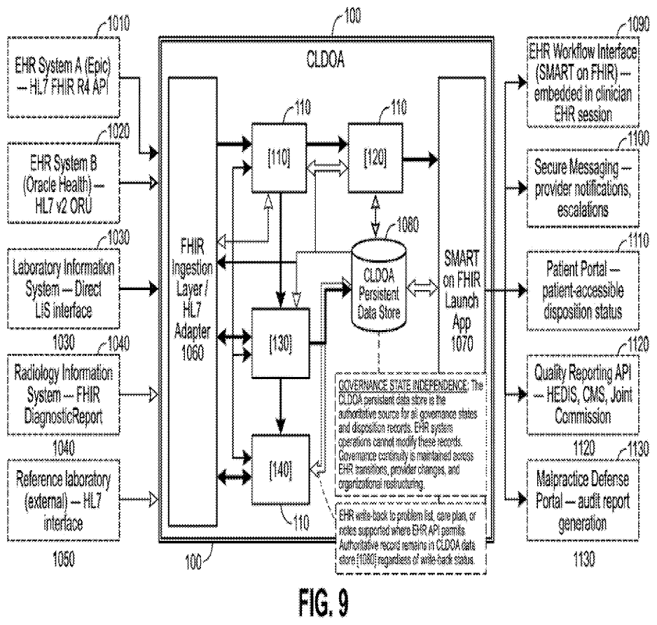

### DETAILED DESCRIPTION OF PREFERRED EMBODIMENTS

### I. Overview

The following detailed description sets forth specific embodiments of the invention. It will be understood by persons skilled in the relevant art that the embodiments described are illustrative and not limiting, and that variations, modifications, and equivalents are within the scope of the claims. Like reference numbers refer to like elements throughout the figures.

The Closed-Loop Diagnostic Oversight Architecture (CLDOA) addresses a class of patient safety failures that are architectural in origin: the absence of any system-level constraint requiring that a reviewing clinician produce a documented disposition for each clinically significant abnormal diagnostic result. The invention imposes this constraint through an independent governance layer that operates externally to any specific EHR system, maintains persistent governance state for each result, and enforces documentation requirements through escalation mechanisms that do not depend on the clinician's voluntary engagement with EHR alert interfaces.

The architecture is organized around four subsystems that interact through defined interfaces: the Disposition Constraint Enforcer (DCE), which classifies results and enforces disposition requirements; the Contextual Decision Support Engine (CDSE), which assembles clinical context at the point of review; the Longitudinal Pattern Governor (LPG), which aggregates result sequences and modifies governance requirements based on pattern strength; and the Audit and Accountability Registry (AAR), which generates and preserves the patient-safety-grade record of all disposition decisions and governance events.

### II. System Architecture and Components

### A. Result Ingestion Layer

The result ingestion layer receives result data from one or more clinical information systems through standardized health information exchange interfaces. In preferred embodiments, the result ingestion layer is configured to receive HL7 FHIR R4 DiagnosticReport and Observation resources from EHR systems that support FHIR API access. In alternative embodiments, the result ingestion layer may receive data through HL7 v2 ORU (Observation Result Unsolicited) messages, through direct laboratory information system interfaces, through radiology information system interfaces, or through any combination of the foregoing.

Upon receipt, each result is assigned a system-generated unique result identifier (URID) and a receipt timestamp. The ingestion layer performs an initial completeness check to confirm that the received result includes the minimum data elements required for classification: result type, result value or coded finding, applicable reference range or normal/abnormal designation, ordering provider identifier, patient identifier, and collection or procedure timestamp. Results that do not satisfy the completeness check are routed to a pending-data queue and are not advanced to classification until the missing elements are received or a data-exception disposition is manually applied.

### B. Result Classification Engine

The result classification engine assigns each result to a disposition tier based on four input categories: result type characteristics, value magnitude relative to reference range, patient-specific contextual factors, and configurable clinical governance rules maintained in a clinical rules repository.

Result type characteristics include the laboratory or imaging modality that produced the result, the analyte or finding type, the reference range applicable to the patient's demographic group (where reference ranges are demographic-specific, including but not limited to pediatric, adult, and geriatric reference ranges, and sex-specific reference ranges), and the clinical classification of the result type as routine, watch-list, time-sensitive, or critical per configurable health system policy.

Value magnitude relative to reference range is computed as a normalized deviation score expressing the result value as a function of the distance from the reference range boundary. In preferred embodiments, the deviation score is computed separately for results falling below the lower reference limit and results exceeding the upper reference limit, and is calibrated against an artifact rate reference dataset to support artifact probability estimation as described below.

Patient-specific contextual factors accessed by the classification engine include: the patient's prior result history for the same test type, including the longitudinal trend of prior values; the patient's current medication list as recorded in the EHR, screened against a drug-effect reference database for agents known to affect the analyte in question; active problem list entries relevant to the result type; and any specimen-handling exception flags associated with the result, including hemolysis indices, collection anomalies, or laboratory quality control flags.

The configurable clinical governance rules repository contains health-system-configured rules that specify: the disposition tier assignment for each combination of result type and deviation magnitude; override rules applicable when patient-specific contextual factors are present; and time-window parameters specifying the maximum elapsed time between result classification and required disposition documentation for each tier.

In preferred embodiments, three disposition tiers are defined as follows. Tier 1 (Watchlist) is assigned to results exhibiting abnormality at a magnitude that is clinically relevant but below the threshold of urgent action, and includes results for which a documented observation, repeat-test order, or watchful waiting rationale constitutes an appropriate disposition. Tier 2 (Follow-up Required) is assigned to results exhibiting abnormality at a magnitude or pattern that requires active clinical management including differential diagnosis documentation, subspecialty referral consideration, or patient notification within a defined time window. Tier 3 (Critical/Urgent) is assigned to results that require immediate clinical action, including results meeting the health system's critical value notification criteria, results indicating findings with a high probability of malignancy, and results indicating conditions with an established time-sensitive treatment window.

### C. Disposition State Machine

The disposition state machine is the core architectural element that distinguishes the invention from prior art EHR alert systems. It maintains a defined governance state for each classified result and enforces state transition requirements that cannot be bypassed by clinician inaction.

The disposition state machine defines the following states: RECEIVED (result received by ingestion layer, not yet classified); CLASSIFIED (disposition tier assigned, disposition requirement clock initiated); PENDING_DISPOSITION (result delivered to reviewing clinician interface, disposition not yet recorded); DISPOSITION_RECORDED (valid disposition record received, pending validation); CLOSED (disposition record validated against tier requirements, result governance complete); ESCALATED (disposition requirement not satisfied within time window, result advanced to higher governance authority); and DATA_EXCEPTION (result failed completeness check, pending data completion).

Critically, the CLOSED state is not accessible by any action other than the receipt of a valid disposition record satisfying the documentation requirements for the assigned tier. There is no clinical-user-accessible action that transitions a result directly from PENDING_DISPOSITION to CLOSED without producing a disposition record. This architectural constraint is the primary mechanism by which the invention prevents silent closure through clinician inaction.

A disposition record, for the purposes of state machine validation, is a structured data object containing: the identity and credential level of the clinician recording the disposition; the timestamp of disposition recording; the disposition category selected from a configurable controlled vocabulary (including at minimum: repeat-test-ordered, referral-placed, watchful-waiting-documented, artifact-assessed-with-rationale, patient-notified-direct-management, and result-incorporated-in-active-management-plan); a free-text or structured rationale field meeting a configurable minimum length requirement for the assigned tier; and for Tier 2 and Tier 3 results, a required next-action specification including the responsible clinician or service and the required completion date.

### D. Constraint Enforcement Module

The constraint enforcement module monitors the disposition state machine and enforces the architectural constraint that results in the CLASSIFIED or PENDING_DISPOSITION state cannot achieve a CLOSED state absent a valid disposition record. The constraint is enforced through the following mechanisms:

First, result-level access controls prevent the modification of a result's governance state to CLOSED through any interface other than the disposition recording interface, including administrative interfaces, EHR write-back interfaces, and batch processing operations. The governance state of each result is maintained in the CLDOA's own persistent data store and is not modifiable by operations directed at the originating EHR system.

Second, the constraint enforcement module validates each disposition record against the documentation requirements for the assigned tier before permitting the state transition to DISPOSITION_RECORDED. Disposition records that do not satisfy all required fields, do not meet minimum rationale content requirements, or are submitted by a clinician who does not possess the credential level required for disposition of the assigned tier are rejected with a specific rejection code and the result remains in PENDING_DISPOSITION state.

Third, the constraint enforcement module enforces the separation of constraint authority from both the EHR system and the reviewing clinician. The constraint authority is vested in the CLDOA governance configuration maintained by the health system's designated governance administrator, and cannot be modified by individual clinicians or by EHR administrative operations. This provider-independence property ensures that the governance constraints remain in force across EHR system changes, provider credentialing changes, and organizational transitions.

### D1. Disposition Record Validation Logic—State Transition Gate

The state transition from PENDING_DISPOSITION to DISPOSITION_RECORDED is mediated by a validation gate that evaluates each submitted disposition record against a multi-factor validation specification. The validation gate implements the following logic, executed atomically upon each disposition record submission:
FUNCTION validate_disposition_record(record, result, tier_config):
// Gate 1: Structural completeness
   IF record. disposition_category IS NULL OR
   record. disposition_category NOT IN
   tier_config. allowed_categories:
      RETURN REJECT(code: INVALID_CATEGORY)
// Gate 2: Rationale content requirement
   IF LENGTH(record. rationale_text)<
   tier_config. min_rationale_chars:
      RETURN REJECT(code: RATIONALE_INSUFFICIENT)
// Gate 3: Next-action specification (Tier 2 and Tier 3 only)
   IF result. tier IN {TIER_2, TIER_3}:
      IF record. next_action. responsible_clinician IS NULL OR
      record. next_action. required_completion_date IS NULL:
         RETURN REJECT(code: NEXT_ACTION_INCOMPLETE)
// Gate 4: Credential authorization
   IF clinician. credential_level<
   tier_config. min_credential_for_tier:
      RETURN REJECT(code: INSUFFICIENT_CREDENTIAL)
// Gate 5: Escalation authority alignment
   IF result. governance_state==ESCALATED:
      IF clinician. role NOT IN
      result. current_escalation_level. authorized_roles:
      RETURN REJECT(code:
      NOT_AUTHORIZED_AT_ESCALATION_LEVEL)
// Gate 6: Temporal consistency
   IF record. timestamp<result. classification_timestamp:
      RETURN REJECT(code: TEMPORAL_INCONSISTENCY)
// All gates passed—permit transition
   RETURN ACCEPT(transition_to: DISPOSITION_RECORDED)

Each rejection code causes the constraint enforcement module to return a structured error response to the disposition interface, specifying the gate that failed and, where applicable, the corrective action required. The clinician may correct the deficiency and resubmit without loss of the partial disposition record. The result remains in PENDING_DISPOSITION state for the duration. Each failed validation attempt, including the rejection code and the submitted record content, is recorded in the Audit and Accountability Registry as a governance event, preserving the complete interaction history for each result.

The validation gate specification is configurable by the health system governance administrator within bounds established by the CLDOA platform. Configurable parameters include the minimum rationale character count by tier (default: 50 characters for Tier 1; 150 characters for Tier 2; 250 characters for Tier 3); the controlled vocabulary of allowed disposition categories by result type; and the credential-level requirements for disposition of each tier. Non-configurable constraints—the structural completeness check, the temporal consistency check, and the escalation authority alignment check—are enforced by the platform and cannot be disabled by governance administrators or individual clinicians.

### E. Contextual Decision Support Engine (CDSE)

The Contextual Decision Support Engine assembles and presents the clinical context required to support a genuinely informed disposition decision at the point of result review. The CDSE is triggered upon delivery of a classified result to the clinician disposition interface and assembles context from multiple clinical data sources prior to interface rendering.

The CDSE assembles the following contextual elements for each result: the patient's longitudinal result history for the same test type, presented as a time-series including the current result, with trend direction and rate-of-change computed and displayed; the artifact probability estimate for the current result as computed by the artifact probability estimation method described below; applicable clinical guidelines and evidence-based recommendations for the result type and magnitude, drawn from a configurable clinical guidelines repository; a consequence table specifying the clinical outcomes associated with delayed or missed diagnosis for the result type, including where available the time-sensitivity of intervention; the patient's current medication list filtered for agents known to affect the relevant analyte, with effect direction and clinical significance indicated; and the minimum documentation requirements for the result's assigned disposition tier.

### F. Artifact Probability Estimation

Artifact probability estimation is a method of the invention for generating a quantitative estimate of the probability that an abnormal result value reflects a true physiologic finding versus a non-physiologic artifact attributable to pre-analytical, analytical, or post-analytical variables.

In preferred embodiments, the artifact probability estimation method comprises: accessing the patient's longitudinal result history for the same test type from the patient record; computing a deviation magnitude score expressing the current result value as a normalized function of the reference range; accessing a calibrated artifact rate reference dataset that provides, for each test type, empirically derived artifact probability estimates as a function of deviation magnitude, stratified by patient demographic group; adjusting the base artifact probability from the reference dataset based on the presence or absence of patient-specific modifying factors including: specimen handling flags associated with the current result; medication list entries for agents with known effects on the analyte; temporal proximity to prior results of similar magnitude indicating a pattern of recurrence that reduces artifact likelihood; and active problem list entries that provide a plausible physiologic explanation for the abnormal value.

The artifact probability estimate is computed as a posterior probability combining the base artifact rate from the reference dataset with the modifying factors, using a configurable Bayesian or rule-based adjustment framework. The estimate is expressed as a probability value between 0.0 and 1.0, with associated confidence intervals derived from the reference dataset sample size, and is presented to the reviewing clinician alongside a plain-language interpretation (e.g., “approximately 8% probability that this value reflects a laboratory artifact given the patient's history and current medications—repeat testing is recommended before attributing to artifact”).

The artifact probability estimate, together with the disposition rationale recorded by the clinician, is preserved in the Audit and Accountability Registry as part of the disposition record. This creates an evidentiary record of the information state at the time of clinical decision that can be used to demonstrate, in subsequent malpractice proceedings or quality review processes, whether the disposing clinician had access to quantitative artifact probability information at the time of the disposition decision.

### G. Longitudinal Pattern Governor (LPG)

The Longitudinal Pattern Governor maintains a persistent longitudinal result thread for each patient that aggregates individual result events across multiple clinical encounters, provider relationships, care settings, and calendar time periods. The LPG applies configurable pattern detection rules to the aggregated result sequence and modifies the governance state and disposition tier requirements of current results based on detected pattern strength.

The longitudinal result thread for a patient is a time-ordered sequence of result records of each type, augmented with metadata including: the governance state and disposition record for each prior result; the clinical setting in which each result was generated; the provider relationship in effect at the time of each result; and any clinical context annotations applied by prior reviewing clinicians. The thread is maintained persistently and does not expire with care episode boundaries, provider transitions, or EHR system changes.

The pattern detection engine applies configurable pattern rules to the longitudinal result thread. Pattern rules are expressed as logical conditions over the properties of the result sequence, including: frequency rules (e.g., N or more results of the same type outside the reference range within a specified time window); trend rules (e.g., result values exhibiting a monotonic downward trend over a specified number of sequential results); persistence rules (e.g., results remaining outside the reference range for a specified cumulative duration regardless of individual visit frequency); and composite rules combining frequency, trend, and persistence conditions.

When a pattern detection rule is satisfied, the LPG takes one or more of the following governance actions: advancing the disposition tier assignment of the current result (e.g., automatically escalating a Tier 1 result to Tier 2 when it represents the third result of the same type outside the reference range within twelve months); generating a pattern notification delivered to the reviewing clinician and, at configurable thresholds, to the patient's designated care coordinator; creating a pattern-based care gap record in the persistent accountability registry; and modifying the required disposition documentation to include a pattern acknowledgment field in which the reviewing clinician documents awareness of the longitudinal result pattern.

The LPG operates across provider and organizational boundaries, maintaining the longitudinal thread across the patient's complete result history available to the health system regardless of which specific provider ordered or reviewed each prior result. This cross-provider, cross-encounter aggregation is the mechanism by which the LPG addresses the temporal fragmentation failure mode described in the background section.

### H. Escalation Engine

The escalation engine monitors the disposition state machine for results in CLASSIFIED or PENDING_DISPOSITION state and initiates escalation actions when the elapsed time since classification exceeds the time-window parameter configured for the assigned disposition tier. Escalation is a state transition to the ESCALATED state and does not extinguish the original disposition requirement; the escalated result remains subject to disposition documentation requirements at the escalated governance authority level.

In preferred embodiments, the escalation hierarchy comprises at least the following levels: (1) primary reviewing clinician; (2) attending physician of record or supervising physician; (3) department chief or medical director; (4) chief quality officer or patient safety officer; and (5) patient notification and patient advocate routing. Health systems may configure additional escalation levels, modify the time-window parameters for each level, and define the notification channels (secure message, EHR task, email, SMS, patient portal) used at each escalation level.

The patient advocate routing at escalation Level 5 constitutes a redundant safety loop that operates outside the clinician hierarchy. When a result remains in ESCALATED state after all configured clinician escalation levels have been exhausted without receipt of a valid disposition record, the escalation engine initiates two concurrent actions: (1) a patient notification through the patient communication pathway, expressed in plain-language format appropriate to the patient's documented health literacy level; and (2) a care coordination work queue entry assigned to the designated patient advocate or care coordinator role for the patient's care setting. The patient advocate work queue entry presents the result type, the plain-language description of the clinical significance, the elapsed time since classification, the names and notification timestamps of all clinicians in the escalation hierarchy who received notifications without recording a disposition, and a pre-drafted patient outreach communication and clinical order suggestion.

The patient advocate routing is structurally distinct from the clinician escalation levels in two respects. First, the patient advocate is not required to record a disposition record satisfying the clinical documentation requirements of the assigned tier; instead, the patient advocate records a care coordination action confirming that the patient has been notified and that follow-up care has been arranged through a mechanism outside the standard clinical workflow. This care coordination record satisfies a distinct set of validation requirements tailored to the patient advocacy role and is preserved in the AAR as a separate record type. Second, the patient advocate routing does not extinguish the original clinical disposition requirement; the result remains in ESCALATED state from the perspective of the clinical governance hierarchy, and the AAR preserves the complete record of clinician non-response for quality review and malpractice defense purposes, while simultaneously ensuring that the patient receives actionable information and care coordination support regardless of clinician engagement.

This redundant safety loop architecture—in which the patient and a non-clinician advocate constitute a final escalation tier that operates in parallel with rather than in series with the clinical hierarchy—is a distinguishing feature of the CLDOA that addresses the failure mode in which all configured clinician escalation levels fail to produce a disposition record. By routing to the patient directly as a governance mechanism, the CLDOA ensures that the patient's ability to seek follow-up care is not dependent on the successful engagement of any individual clinician or administrative authority within the health system's governance hierarchy.

For Tier 3 (Critical/Urgent) results, the escalation engine initiates concurrent notification to the primary reviewing clinician and the attending of record simultaneously upon classification, without awaiting expiration of a time window. For Tier 3 results that involve findings with a high probability of malignancy or other time-sensitive diagnoses, the escalation engine additionally initiates patient notification through the patient communication pathway after a configurable short time window if no disposition record has been received from any clinician in the escalation hierarchy.

### I. Audit and Accountability Registry (AAR)

The Audit and Accountability Registry is a patient-safety-grade persistent data store that preserves an immutable record of all governance events associated with each result, including result receipt, classification, disposition delivery, disposition recording, validation outcomes, escalation events, and patient notification events.

For each governance event, the AAR records: the event type and timestamp; the result identifier and patient identifier; the governance state before and after the event; the identity and credential level of any human actor who initiated or completed the event; the complete content of any disposition record received, including all fields, the rationale text, and the artifact probability estimate displayed at the time of recording; the escalation level in effect at the time of any disposition recording; and any system-generated notifications delivered in connection with the event.

The AAR is designed to support three primary use cases: malpractice defense documentation (providing evidence of whether a reviewing clinician had access to the relevant clinical context, the artifact probability estimate, and the disposition requirement at the time of a challenged clinical decision); quality and performance improvement reporting (providing result-level and provider-level follow-up compliance metrics for health system quality programs); and regulatory compliance documentation (providing the structured records required by Joint Commission standards for critical value communication and abnormal result follow-up, CMS quality measure documentation, and state-specific medical record retention requirements).

The AAR is implemented as an append-only data store with cryptographic integrity protection. Records in the AAR cannot be deleted, modified, or overwritten by any user-level operation, including administrative operations. A complete audit log of all AAR access operations, including read operations, is maintained and is itself an AAR record.

### J. EHR Independence and Integration Architecture

A defining property of the CLDOA is that its constraint enforcement authority is independent of any specific EHR system. The CLDOA maintains its own persistent result governance state, its own disposition documentation records, and its own escalation hierarchy configuration in data stores under its own control. EHR system operations cannot directly modify CLDOA governance states or disposition records.

Integration with EHR systems is achieved through standardized health information exchange interfaces. In preferred embodiments, EHR systems deliver results to the CLDOA via HL7 FHIR R4 DiagnosticReport push notifications or polling. The CLDOA delivers disposition interface content to clinical users through EHR-embedded SMART on FHIR launch applications that render within the EHR workflow without requiring EHR-side configuration of governance rules. Write-back of disposition records to the EHR problem list, care plan, or clinical notes sections is supported through the EHR's write-back API where available, but the authoritative disposition record is maintained in the AAR regardless of EHR write-back status.

The EHR independence property ensures that the governance constraints remain in force when a health system transitions between EHR platforms, when patients receive care at multiple facilities using different EHR systems, and when providers change employment or practice affiliation. The CLDOA's persistent longitudinal result threads continue to accumulate result data through EHR transitions, maintaining governance continuity that EHR-native result management systems cannot provide.

### III. Physician-Assistive Design Architecture

### A. Pre-drafted Disposition Records

A fundamental design principle of the CLDOA is that the system reduces the cognitive and time burden on reviewing clinicians by generating pre-drafted disposition records that clinicians confirm or modify, rather than requiring clinicians to compose disposition documentation from scratch. This confirm-not-compose design principle is implemented through a Guideline Mapping Engine and a Disposition Drafting Engine operating as subsystems of the Contextual Decision Support Engine.

The Guideline Mapping Engine selects the applicable clinical guideline recommendation for a classified result by matching the result type, the deviation magnitude tier, and the patient's demographic profile against a configurable clinical guidelines repository. The guidelines repository is maintained by the health system's clinical governance administrator and contains structured representations of published clinical guidelines for each result type covered by the system, including guideline source, evidence grade, applicable demographic scope, and recommended next actions expressed as structured data objects rather than free text. In preferred embodiments, the guidelines repository is updated through a managed content subscription service and versioned so that the guideline version in effect at the time of each disposition decision is preserved in the Audit and Accountability Registry.

The Disposition Drafting Engine generates a candidate disposition record pre-populated with at least: the guideline-recommended next action expressed as a structured disposition category from the controlled vocabulary; the recommended time window for the next action derived from the applicable guideline; the responsible clinician role inferred from the result type and the patient's current care team; a plain-language rationale statement synthesized from the assembled contextual elements including the artifact probability estimate, the relevant prior result trend, and the applicable guideline recommendation; and, for Tier 2 and Tier 3 results, a next-action specification pre-populated with the recommended follow-up action and deadline.

The pre-drafted disposition record is presented to the reviewing clinician in the disposition interface with each pre-populated field visible and editable. The reviewing clinician may confirm the pre-drafted record as presented, modify any field before confirmation, or reject the pre-draft and compose an alternative disposition record. Confirmation of an unmodified pre-drafted record constitutes a valid disposition record satisfying all documentation requirements for the assigned disposition tier. The confirmed or modified record is stored in the Audit and Accountability Registry with a flag indicating whether each field was confirmed as pre-drafted or modified by the clinician.

For guideline-concordant dispositions in straightforward Tier 1 cases—representing the majority of classified results in a typical health system deployment—the pre-drafted confirmation workflow requires a single review-and-confirm interaction estimated at fewer than thirty seconds, including the clinician's review of the assembled contextual panel. This design eliminates the requirement that clinicians compose free-text rationale documentation from memory under time pressure, which is identified in the clinical literature as a primary barrier to complete abnormal result documentation in high-volume ambulatory settings.

### B. Tiered Batch Review Interface

The CLDOA provides a tiered batch review interface that organizes pending classified results into a prioritized queue enabling clinicians to process multiple low-acuity results in rapid sequential succession while concentrating deliberate individual review on high-acuity results.

The priority queue orders pending results by a composite priority score computed from at minimum: the result's disposition tier; the elapsed time since classification relative to the tier's time-window parameter; the detected longitudinal pattern strength for the patient; and a configurable urgency weighting applied to specific result types by health system policy. The priority queue presents Tier 3 and high-elapsed-time Tier 2 results at the top of the queue for individual review. Tier 1 results for which a pre-drafted disposition has been generated and no modifying factors are present are grouped into a batch review panel presented below individual-review results.

In the batch review panel, each Tier 1 result is presented as a compact card displaying: the result type and value; the key contextual summary including the artifact probability estimate and the most clinically relevant prior value; the pre-drafted disposition summary; and a confirmation control. The clinician may confirm the pre-drafted disposition for each card with a single interaction, advance to review the full contextual panel for any card before confirming, or flag any card for deferred individual review. The batch review panel does not require the clinician to navigate to individual result detail views for straightforward confirmations, reducing the per-result interaction to a review-and-confirm sequence rather than a navigate-review-compose-submit sequence.

The batch review interface is designed to make the processing of guideline-concordant Tier 1 results comparable in time efficiency to existing EHR alert dismissal workflows, while producing valid disposition records that satisfy governance requirements. This parity of time cost between compliant documentation and non-compliant dismissal is a design objective of the batch interface and a key mechanism by which the system achieves adoption without requiring clinicians to accept increased workload as a precondition of compliance.

### C. Guideline-driven Suggestion Engine

The Guideline-Driven Suggestion Engine is a subsystem of the CDSE that selects, retrieves, and presents the applicable clinical practice guideline recommendation for each classified result at the point of clinical review. The Suggestion Engine eliminates the requirement that reviewing clinicians recall guideline criteria from memory during result review, which is a documented source of guideline non-adherence in high-volume clinical settings where clinicians encounter diverse result types across multiple specialties.

The Suggestion Engine operates by: mapping each classified result to one or more applicable guidelines in the clinical guidelines repository based on result type, result value, and patient demographic; retrieving the specific recommendation applicable to the result's deviation magnitude and the patient's relevant clinical characteristics; expressing the recommendation as a structured next-action specification; and presenting the recommendation in the disposition interface alongside the plain-language guideline citation, the evidence grade, and the recommended time window. The Suggestion Engine additionally identifies and flags any patient-specific factors that may modify the standard guideline recommendation, including comorbidities, medications, and prior result history, and presents a modifier notification when such factors are detected.

In preferred embodiments, the Suggestion Engine maintains awareness of guideline version currency and notifies the health system governance administrator when a guideline in the repository has been superseded by a more recent version, enabling governance administrators to update the repository before clinicians receive outdated recommendations. The guideline version presented to each clinician is recorded in the Audit and Accountability Registry as part of the disposition record, creating an evidentiary record that the clinician received the then-current guideline recommendation at the time of the disposition decision.

### D. Proactive Patient Surveillance and Worry-List Surfacing

The CLDOA provides a proactive patient surveillance function that identifies patients with unresolved longitudinal abnormal result patterns and surfaces them to the reviewing clinician or care coordinator for outreach, independent of whether a new result has been received for the patient. This function addresses the documented clinical burden of the informal physician worry list—the mental inventory of patients with prior ambiguous or borderline findings who may warrant follow-up that has not been generated through the normal result delivery workflow.

The proactive surveillance function operates through a Pattern Monitoring Engine that periodically evaluates the longitudinal result threads of all patients under a clinician's care, applying configurable surveillance rules that identify patients meeting criteria for proactive outreach. Surveillance rules include at minimum: patients with a prior Tier 2 result whose required follow-up action was documented but for which no follow-up result has been received within the guideline-specified window; patients with a longitudinal result pattern of borderline-but-individually-below-threshold values that, considered as a sequence, satisfy a composite pattern rule; and patients with a documented incidental finding for which guideline-specified follow-up imaging has not been ordered within the required window, where no new triggering result has arrived through the normal ingestion pathway.

The output of the proactive surveillance function is a care gap work queue presented to the reviewing clinician or care coordinator as a separate view from the active result disposition queue. Each entry in the care gap work queue presents: the patient identifier and the relevant clinical summary; the prior result or pattern that triggered the surveillance flag; the time elapsed since the flag-triggering event; the applicable guideline recommendation for the surveillance situation; and a pre-drafted outreach action including a suggested patient communication and a suggested order or referral. The clinician or care coordinator may confirm the pre-drafted outreach action, modify it, or document a reason for deferral. As with disposition records, the outreach action record is preserved in the Audit and Accountability Registry.

The proactive surveillance function structurally externalizes the cognitive burden of maintaining a mental worry list by converting an informal and fallible human memory function into a persistent, systematic, and auditable system function. Clinicians who use the proactive surveillance queue are relieved of the cognitive overhead of tracking borderline patients between visits while simultaneously receiving protection against the liability exposure associated with undocumented follow-up gaps.

### E. Physician Workload Analytics

The CLDOA provides workload analytics that give reviewing clinicians and clinical administrators visibility into the distribution of result disposition burden across the clinical staff, the proportion of results processed through pre-drafted confirmation versus composed documentation, and the average time-per-disposition by result tier and result type. These analytics serve two functions: they enable health system administrators to identify and address workload distribution inequities in result review assignments; and they provide clinicians with a transparent record of their own result governance performance that can be used for professional development, quality improvement, and credentialing purposes.

In preferred embodiments, workload analytics are presented to individual clinicians as a personal dashboard accessible through the disposition interface, showing their current pending result queue depth, their rolling average time-per-disposition by tier, their pre-drafted confirmation rate, and a comparison of their follow-up compliance metrics against configurable peer benchmarks. The personal dashboard is designed to provide clinicians with actionable information about their own workflow patterns rather than to function as a supervisory monitoring tool, and access to individual clinician analytics is governed by role-based access controls that restrict aggregate comparisons to authorized quality improvement personnel.

### IV. EXEMPLARY EMBODIMENTS

### A. Hematologic Abnormality—Isolated Neutropenia

In an exemplary embodiment, a patient presents to a primary care physician for a routine physical examination. A complete blood count (CBC) with differential is ordered. The CBC result, which includes a white blood cell count of 2.1×10⁹/L (reference range 4.5-11.0×10⁹/L), is delivered by the laboratory information system to the CLDOA ingestion layer as an HL7 FHIR DiagnosticReport resource.

The result classification engine computes a deviation magnitude score based on the WBC value and the applicable reference range. The classification engine accesses the patient's longitudinal CBC history, which includes WBC values of 4.2, 3.8, and 4.0 over the preceding eighteen months, indicating a downward trend. The classification engine accesses the patient's medication list and identifies no myelosuppressive agents. The classification engine assigns the result to Tier 2 (Follow-up Required) based on the combination of the magnitude of the abnormality, the downward trend in prior values, and configurable health system rules specifying Tier 2 assignment for WBC values below 2.5×10⁹/L in the absence of a documented prior diagnosis of chronic neutropenia.

The CDSE assembles the following contextual panel for delivery to the reviewing clinician: the current WBC value alongside a time-series chart of the four prior WBC values showing the downward trend; an artifact probability estimate of 8.2% (reflecting the relatively low artifact probability at a WBC deviation of this magnitude in the absence of specimen handling flags); applicable ACP guideline recommendations specifying repeat CBC with differential within 2-4 weeks for a confirmed isolated neutropenia at this count; a consequence table noting that isolated neutropenia in this demographic warrants consideration of autoimmune, drug-induced, and early hematologic malignancy etiologies; and the Tier 2 disposition requirement specifying that the disposition record must include a differential diagnosis statement and a next-action specification with a responsible clinician and completion date.

The reviewing clinician, presented with this assembled context and the quantitative artifact probability estimate, documents a disposition record specifying: “WBC 2.1—artifact probability per system 8%; prior trend downward over 18 months; differential includes benign ethnic neutropenia, drug-induced (medications reviewed—no myelosuppressives), autoimmune, early hematologic malignancy. Plan: repeat CBC with differential in 3 weeks. Responsible: primary care. If confirmed neutropenia, hematology referral.” The disposition record is validated against Tier 2 requirements and the result transitions to CLOSED state.

Three weeks later, a repeat CBC is ordered. If the repeat CBC also shows a WBC below 2.5×10⁹/L, the LPG detects a pattern satisfying the frequency rule for two consecutive below-threshold CBCs within a twelve-month window and automatically advances the disposition tier of the repeat result to Tier 2 with a modified disposition requirement that includes a hematology referral field. If no repeat CBC is ordered within the four-week window specified in the prior disposition record, the escalation engine generates a notification to the reviewing clinician and, if unresolved, to the attending of record.

### B. Incidental Pulmonary Nodule

In another exemplary embodiment, an incidental pulmonary nodule measuring 8 mm is identified in a radiology report from a CT scan ordered for an unrelated indication. The radiology report is delivered to the CLDOA ingestion layer as an HL7 FHIR DiagnosticReport resource with a structured finding code indicating a pulmonary nodule and a size measurement.

The classification engine assigns the finding to Tier 2 (Follow-up Required) based on the nodule size (8mm, above the 6 mm threshold configurable as the Tier 2 boundary for solid pulmonary nodules in patients over 35 years of age per Fleischner Society guidelines) and the absence of any prior documented pulmonary nodule management plan in the patient record. The CDSE presents the applicable Fleischner Society guideline recommendation (CT follow-up at 3-6 months for solid nodules 6-8mm in a patient with a smoking history, or 6-12 months for a patient without a smoking history), the patient's smoking status from the problem list, and the Tier 2 disposition requirement specifying a next-action specification including the imaging order and responsible clinician.

The LPG monitors for placement of the follow-up CT order within the Fleischner-specified window. If the follow-up CT order is not placed within the disposition-specified window, the escalation engine advances governance to the attending of record and, at a configurable secondary threshold, to the quality officer. If the follow-up CT is ordered and performed and reveals no change, the new result is classified at an updated tier consistent with Fleischner guidelines for stable nodules at follow-up. If the follow-up CT reveals interval growth, the new result is classified at Tier 3 and the escalation engine initiates concurrent notification to the ordering clinician and attending of record.

### C. Rising PSA Velocity Across Multiple Providers

In another exemplary embodiment, a 64-year-old male patient undergoes annual prostate-specific antigen (PSA) screening over a period of three years. PSA results are ingested by the CLDOA ingestion layer from three separate laboratory information systems corresponding to three different clinical encounters with three different primary care providers within the same health system. The result values are: 2.8 ng/mL (month 0), 3.6 ng/mL (month 12), 4.5 ng/mL (month 24), and 5.9 ng/mL (month 36). Each individual result, considered in isolation, falls in a range that different reviewing clinicians have historically managed with watchful waiting rather than immediate referral. No single reviewing provider has seen more than one prior value because each encounter involved a different provider relationship.

Upon receipt of the fourth PSA result at month 36, the LPG retrieves the complete longitudinal PSA thread for the patient, aggregating all four values across the three provider relationships and three separate care episodes. The pattern detection engine evaluates the sequence against configured pattern rules and identifies satisfaction of two rules simultaneously: (1) a trend rule specifying a monotonic upward trend over four sequential PSA results; and (2) a velocity rule specifying that PSA velocity of 1.0 ng/mL/year or greater in a patient with PSA values between 2.0 and 10.0 ng/mL constitutes a pattern warranting urologic evaluation per applicable AUA guidelines. The computed PSA velocity is 1.03 ng/mL/year over the 36-month period. Neither rule would have been satisfied by any two or three consecutive results in isolation; the pattern is visible only in the complete four-point longitudinal thread aggregated across provider boundaries.

The LPG automatically advances the disposition tier of the fourth PSA result from Tier 1 (which would have been assigned based on the individual result value alone) to Tier 2, and generates a pattern notification. The CDSE assembles a contextual panel presenting: the time-series chart of all four PSA values with computed velocity annotation; the applicable AUA guideline recommendation for PSA velocity exceeding 0.75 ng/mL/year; the provider identifiers for each prior result encounter; and the pre-drafted disposition record specifying urology referral as the recommended next action. The reviewing clinician for the fourth result—who would not otherwise have had access to the three prior values from other providers—is presented with the complete longitudinal picture at first result presentation, without requiring manual chart review across three separate encounter records.

This embodiment illustrates the cross-provider pattern detection capability of the LPG as the primary mechanism for addressing temporal fragmentation of result sequences across care episodes. The clinical significance of the PSA velocity pattern in this embodiment is not detectable from any single result or any two-point sequence from a single provider relationship; it requires the aggregated four-point longitudinal thread. The CLDOA governance architecture surfaces this pattern at the point of clinical decision for the fourth result, converting a cross-provider pattern that would otherwise exist only in the aggregate medical record into an actionable governance requirement at the time of result review.

### D. HSIL Abnormal Cervical Cytology With Provider Coverage Transition

In another exemplary embodiment, a 34-year-old female patient undergoes a Papanicolaou (Pap) smear during a routine well-woman examination. The cervical cytology result, indicating high-grade squamous intraepithelial lesion (HSIL), is delivered to the CLDOA ingestion layer as an HL7 FHIR DiagnosticReport resource. The classification engine assigns the result to Tier 2 (Follow-up Required) based on the HSIL finding code and the applicable ASCCP 2019 risk-based management guideline, which specifies expedited treatment or colposcopy referral for HSIL findings. The time-window parameter for Tier 2 results of this category is configured to 14 calendar days at the deploying health system.

The result is delivered to the CLDOA disposition interface and its governance state transitions to PENDING_DISPOSITION. The ordering provider is on approved leave, and the result is routed to a covering provider's disposition queue. The covering provider reviews the result, notes the HSIL finding, and intends to place a colposcopy referral. Due to an interruption in the clinical workflow, the covering provider navigates away from the disposition interface without completing the disposition record. The result governance state remains PENDING_DISPOSITION—it does not transition to CLOSED state because no valid disposition record has been received. This behavior is the direct consequence of the architectural constraint enforced by the DCE: the governance state is maintained in the CLDOA's persistent data store independently of the covering provider's inbox state, and the result cannot achieve a closed state through any mechanism other than receipt of a validated disposition record.

When the ordering provider returns from leave and resumes clinical duties, the result does not appear in the ordering provider's inbox as a new item—it was delivered during the coverage period. However, the result remains in PENDING_DISPOSITION state in the CLDOA governance layer, and the 14-day time window has elapsed without a disposition record. The escalation engine has advanced governance to Level 2 (attending of record) and subsequently to Level 3 (department chief) in accordance with the configured escalation hierarchy. The CLDOA generates notifications to both the attending of record and the department chief, each presenting the assembled contextual panel including the HSIL finding, the ASCCP guideline recommendation, the elapsed time since classification, and the pre-drafted colposcopy referral disposition record. Upon receipt of the department chief's notification, a colposcopy referral is placed and the disposition record is validated against Tier 2 requirements. The result transitions to CLOSED state with the complete escalation history preserved in the AAR.

This embodiment illustrates three architectural properties of the CLDOA operating in combination. First, the provider-independence of the governance state: the coverage transition does not reset or extinguish the disposition requirement, which remains in force regardless of changes in the provider relationship. Second, the EHR-independence of the governance authority: the result's absence from the returning ordering provider's EHR inbox does not affect the CLDOA governance state, which is maintained in the CLDOA's own persistent data store and is not dependent on EHR inbox state. Third, the escalation engine's systematic advancement through the governance hierarchy in the absence of disposition documentation, which ensures that a clinically urgent finding that falls through a coverage transition does not remain unaddressed indefinitely. The AAR preserves the complete escalation timeline, providing an evidentiary record of the governance history for malpractice defense and quality review purposes.

What is claimed is:

1. A computer-implemented system for enforcing documented clinical disposition of abnormal diagnostic results, the system comprising:
a) one or more processors;
b) one or more non-transitory computer-readable media storing instructions that, when executed by the one or more processors, cause the system to:
c) receive, from one or more clinical information systems via a health information exchange interface, diagnostic result data comprising at least a result type, a result value, an applicable reference range, a patient identifier, and an ordering clinician identifier;
d) classify each received result into a disposition tier from a plurality of disposition tiers based at least on the result type, a deviation magnitude computed as a function of the result value relative to the applicable reference range, and one or more patient-specific contextual factors retrieved from a patient record associated with the patient identifier;
e) maintain, in a persistent data store separate from the one or more clinical information systems, a governance state for each classified result;
f) enforce a constraint that prevents a result's governance state from transitioning to a closed state absent receipt of a disposition record that satisfies documentation requirements associated with the result's disposition tier, wherein the constraint is enforced independently of any operation directed at the one or more clinical information systems and cannot be satisfied by clinician inaction alone; and
g) initiate escalation of a result's governance to a higher authority level when a disposition record satisfying the documentation requirements has not been received within a time window associated with the result's disposition tier.

2. The system of claim 1, wherein the patient-specific contextual factors comprise one or more of: a longitudinal history of prior results of the same result type for the patient; a current medication list for the patient; active problem list entries for the patient; and specimen handling exception flags associated with the received result.

3. The system of claim 1, wherein the instructions further cause the system to: generate an artifact probability estimate for a received result, the artifact probability estimate expressing a probability that the result value reflects a non-physiologic artifact rather than a true physiologic finding; wherein the artifact probability estimate is computed as a function of at least the deviation magnitude, a reference artifact rate for the result type at the deviation magnitude derived from a calibrated reference dataset, and one or more patient-specific modifying factors; and present the artifact probability estimate to a reviewing clinician concurrently with the documentation requirements for the result's disposition tier.

4. The system of claim 3, wherein the patient-specific modifying factors used to compute the artifact probability estimate comprise one or more of: the presence or absence of specimen handling exception flags associated with the result; the presence or absence of medications known to affect the relevant analyte; and the temporal pattern of prior results of the same type in the patient's longitudinal history.

5. The system of claim 1, wherein the documentation requirements for a first disposition tier require at minimum a disposition category selection and a free-text rationale of a minimum configured length; and wherein the documentation requirements for a second disposition tier, representing a higher severity classification than the first disposition tier, further require a next-action specification comprising a responsible clinician identifier and a required completion date.

6. The system of claim 1, wherein initiating escalation comprises generating a notification to at least one of: a supervising physician; a department chief or medical director; a chief quality officer; or a patient-facing communication channel; and wherein the escalation does not extinguish the disposition documentation requirement but instead extends the requirement to the escalated authority level.

7. The system of claim 1, wherein the constraint that prevents transition to a closed state is enforced through access controls that prevent modification of the governance state through any interface other than a disposition recording interface, including administrative interfaces and operations directed at the one or more clinical information systems.

8. The system of claim 1, wherein the health information exchange interface comprises one or more of: an HL7 FHIR R4 DiagnosticReport subscription or polling interface; an HL7 v2 ORU message interface; a laboratory information system interface; and a radiology information system interface.

9. A computer-implemented method for generating artifact probability estimates for abnormal diagnostic results at the point of clinical review, the method comprising:
a) receiving an abnormal diagnostic result comprising a result type, a result value, and an applicable reference range;
b) computing a deviation magnitude score as a normalized function of the result value's deviation from the applicable reference range;
c) accessing a calibrated artifact rate reference dataset that provides empirically derived artifact probability estimates for the result type as a function of deviation magnitude;
d) retrieving, from a patient record, one or more patient-specific factors comprising at least a longitudinal history of prior results of the same result type and a current medication list;
e) computing a patient-specific artifact probability estimate by adjusting the artifact rate from the calibrated reference dataset based on the patient-specific factors; and
f) presenting the patient-specific artifact probability estimate to a reviewing clinician at a disposition interface rendered at the time of result review, wherein the artifact probability estimate is displayed alongside a documentation requirement for the result.

10. The method of claim 9, wherein computing the patient-specific artifact probability estimate further comprises applying an adjustment for the temporal pattern of prior results of the same type, wherein a pattern of recurrence of similar abnormal values in the patient's longitudinal history reduces the artifact probability estimate relative to the base rate from the calibrated reference dataset.

11. The method of claim 9, further comprising: storing the artifact probability estimate, together with the disposition record produced by the reviewing clinician, in an audit registry as a timestamped record of the information available to the reviewing clinician at the time of the disposition decision.

12. The method of claim 9, wherein the artifact probability estimate is expressed as a probability value between 0.0 and 1.0 with an associated confidence interval, and wherein a plain-language interpretation of the probability value is generated and displayed to the reviewing clinician.

13. A computer-implemented system for governing clinical follow-up of abnormal diagnostic results across longitudinal care episodes, the system comprising:
a) one or more processors;
b) one or more non-transitory computer-readable media storing instructions that, when executed by the one or more processors, cause the system to:
c) maintain, for each patient, a longitudinal result thread that aggregates result events for the same result type across a plurality of clinical encounters, provider relationships, and care settings, wherein the longitudinal result thread persists across care episode boundaries and is not reset upon changes in the patient's provider relationships;
d) apply a pattern detection rule engine to the longitudinal result thread, wherein the pattern detection rule engine identifies clinically significant patterns based on configurable rules comprising one or more of: a frequency rule specifying a threshold number of results outside the reference range within a time window; a trend rule specifying a monotonic directional change in result values over a sequence of results; a persistence rule specifying a minimum cumulative duration of abnormal result values; and a composite rule combining two or more of the foregoing;
e) upon detection of a clinically significant pattern, automatically advance the disposition tier assignment of a current result for the same result type relative to the tier that would have been assigned absent the detected pattern; and
f) generate a pattern notification delivered to the reviewing clinician that presents the detected pattern in the context of the current result.

14. The system of claim 13, wherein maintaining the longitudinal result thread comprises aggregating result events received from a plurality of different clinical information systems, wherein the aggregation is performed across clinical information systems used by different healthcare organizations that have a treatment relationship with the patient.

15. The system of claim 13, wherein the instructions further cause the system to: generate a pattern-based care gap record in a persistent accountability registry upon detection of a clinically significant pattern, wherein the care gap record comprises the detected pattern description, the pattern rule satisfied, the patient identifier, and the timestamp of detection; and wherein the care gap record persists in the accountability registry regardless of subsequent disposition decisions.

16. The system of claim 13, wherein the pattern notification presented to the reviewing clinician includes: a time-series representation of the relevant prior result values; the specific pattern rule satisfied; and a modified disposition documentation requirement that includes a pattern acknowledgment field in which the reviewing clinician documents awareness of the longitudinal result pattern.

17. A computer-implemented system for ensuring patient notification and engagement in the governance of unresolved abnormal diagnostic results, the system comprising:
a) one or more processors;
b) one or more non-transitory computer-readable media storing instructions that, when executed by the one or more processors, cause the system to:
c) maintain a governance state for each abnormal diagnostic result that has been classified and delivered to a reviewing clinician;
d) generate, upon expiration of a configurable time window without receipt of a disposition record satisfying the documentation requirements for the result's disposition tier, a patient notification comprising a plain-language description of the abnormal finding, the clinical governance status of the finding, and instructions for the patient to request follow-up through a patient-accessible channel;
e) provide a patient-accessible disposition status interface through which the patient may view the current governance state of their results; and
f) route unresolved results to a designated patient advocate or care coordinator when clinician disposition requirements remain unmet beyond a configurable secondary escalation threshold.

18. The system of claim 17, wherein the patient notification is generated only after escalation to the attending of record has failed to produce a disposition record within a configurable time window, such that patient notification is preceded by at least one clinician-level escalation attempt.

19. The system of claim 17, wherein the patient-accessible disposition status interface provides the patient with the ability to: view the current governance state of all abnormal results classified within a configurable lookback period; view the content of any disposition records recorded for each result; and submit an inquiry to the reviewing clinician or care coordinator regarding an unresolved result.

20. The system of claim 17, wherein the patient notification is generated in a reading-level-appropriate format based on a configured patient literacy profile and is delivered through one or more of: a patient portal message; an SMS message; an automated voice notification; or a postal notification.

21. A computer-implemented system for generating patient-safety-grade audit records of clinical disposition decisions for abnormal diagnostic results, the system comprising:
a) one or more processors;
b) one or more non-transitory computer-readable media storing instructions that, when executed by the one or more processors, cause the system to:
c) upon each governance event associated with an abnormal diagnostic result, record in an append-only persistent data store an audit record comprising at minimum: the event type and timestamp; the result identifier and patient identifier; the governance state before and after the event; the identity and credential level of any clinician who initiated or completed the event; and the complete content of any disposition record received;
d) for each disposition record audit entry, additionally record: the artifact probability estimate displayed to the reviewing clinician at the time of disposition recording; the clinical context elements assembled and presented to the reviewing clinician at the time of disposition recording; and the escalation level in effect at the time of disposition recording; and
e) prevent modification, deletion, or overwriting of any audit record through any user-level or administrative operation, and maintain a complete access log of all read and write operations directed at the audit data store.

22. The system of claim 21, wherein the audit record supports generation of at least: a malpractice defense documentation report that, for a specified result and patient, presents the complete governance history, the clinical context presented to each reviewing clinician, and the disposition records and rationales recorded at each governance event; a provider-level follow-up compliance report presenting disposition completion rates and time-to-disposition metrics by provider and result type; and a critical value communication report meeting Joint Commission documentation standards.

23. The system of claim 1, wherein the system operates as a governance layer independent of any specific electronic health record system, and wherein the governance state, disposition records, and audit records maintained by the system are not modifiable by operations directed at any electronic health record system.

24. The method of claim 9, wherein the method is performed by a system operating as a governance layer independent of any specific electronic health record system, and wherein the artifact probability estimates and audit records generated by the method are not modifiable by operations directed at any electronic health record system.

25. The system of claim 13, wherein the system operates as a governance layer independent of any specific electronic health record system, and wherein the longitudinal result threads and pattern detection records maintained by the system are not modifiable by operations directed at any electronic health record system.

26. The system of claim 17, wherein the system operates as a governance layer independent of any specific electronic health record system, and wherein the patient notification records and governance states maintained by the system are not modifiable by operations directed at any electronic health record system.

27. The system of claim 21, wherein the audit data store operates independently of any specific electronic health record system, and wherein the audit records are not modifiable by operations directed at any electronic health record system.

28. A computer-implemented system for generating pre-drafted clinical disposition records for abnormal diagnostic results, the system comprising:
a) one or more processors;
b) one or more non-transitory computer-readable media storing instructions that, when executed by the one or more processors, cause the system to:
c) receive a classified abnormal diagnostic result having an assigned disposition tier and a set of assembled contextual elements comprising at least a patient longitudinal result history, an artifact probability estimate, and one or more applicable clinical guideline recommendations;
d) select, from a clinical guidelines repository, the applicable guideline recommendation for the result based on the result type, the deviation magnitude, and the patient demographic profile;
e) generate a pre-drafted disposition record pre-populated with at least: a disposition category derived from the guideline recommendation; a plain-language rationale statement synthesized from the assembled contextual elements; a next-action specification comprising a recommended follow-up action and a deadline derived from the guideline; and a responsible clinician role inferred from the result type and the patient's current care team;
f) present the pre-drafted disposition record to a reviewing clinician in a disposition interface with all pre-populated fields visible and individually editable; and
g) accept a single confirmation interaction from the reviewing clinician as a complete and valid disposition record satisfying all documentation requirements for the assigned disposition tier, wherein no additional free-text composition is required from the clinician when the pre-drafted record is confirmed without modification.

29. The system of claim 28, wherein the pre-drafted disposition record is generated in fewer than five seconds of result classification, and wherein the pre-drafted record is available to the reviewing clinician at the moment of first result presentation without requiring a separate clinician request.

30. The system of claim 28, wherein the instructions further cause the system to: store, in an audit registry, a record of each confirmed disposition that indicates for each field of the disposition record whether the field was confirmed as pre-drafted or modified by the clinician, enabling retrospective analysis of guideline concordance rates and clinician modification patterns.

31. The system of claim 28, wherein the clinical guidelines repository is versioned, and wherein the version of the applicable guideline in effect at the time of each pre-drafted disposition generation is recorded in the audit registry as part of the disposition record.

32. The system of claim 28, wherein the Guideline Mapping Engine identifies patient-specific factors that modify the standard guideline recommendation, and wherein the pre-drafted disposition record includes a modifier notification presented to the reviewing clinician when such factors are detected, with the modified recommendation expressed in the pre-drafted record.

33. A computer-implemented system for tiered batch processing of classified abnormal diagnostic results, the system comprising:
a) one or more processors;
b) one or more non-transitory computer-readable media storing instructions that, when executed by the one or more processors, cause the system to:
c) maintain a priority queue of pending classified results ordered by a composite priority score computed from at least the result's disposition tier, the elapsed time since classification relative to the tier's required disposition time window, and a detected longitudinal pattern strength for the patient;
d) present high-acuity results comprising Tier 3 results and Tier 2 results with elapsed time exceeding a configurable threshold as individual review items requiring dedicated clinician attention;
e) present Tier 1 results for which a pre-drafted disposition record has been generated and no modifying factors are present as a batch review panel comprising compact result cards, wherein each card displays a contextual summary, the pre-drafted disposition summary, and a single-interaction confirmation control; and
f) accept a single confirmation interaction per card as a complete valid disposition record for a Tier 1 result, without requiring the reviewing clinician to navigate to an individual result detail view or compose free-text documentation, when confirming an unmodified pre-drafted record.

34. The system of claim 33, wherein the batch review panel is presented in a single scrollable view that does not require navigation between screens for sequential processing of multiple Tier 1 results, and wherein the clinician may process a plurality of Tier 1 results without leaving the batch review panel view.

35. The system of claim 33, wherein the instructions further cause the system to: track, for each reviewing clinician, the distribution of time spent on individual-review results versus batch-review results; and present workload analytics to the clinician showing the aggregate time savings attributable to pre-drafted batch processing relative to composed individual documentation.

36. The system of claim 33, wherein each compact result card in the batch review panel provides a one-interaction access path to the full contextual panel and pre-drafted disposition interface for the corresponding result, enabling the reviewing clinician to escalate any batch-panel result to individual review without loss of the pre-drafted record.

37. A computer-implemented system for proactive identification of patients with unresolved longitudinal abnormal result patterns requiring clinical outreach, the system comprising:
a) one or more processors;
b) one or more non-transitory computer-readable media storing instructions that, when executed by the one or more processors, cause the system to:
c) periodically evaluate longitudinal result threads for a plurality of patients under a clinician's care, applying configurable surveillance rules to identify patients meeting criteria for proactive outreach independent of whether a new abnormal result has been received for each patient;
d) generate a care gap work queue comprising patients identified by the surveillance rules, wherein each entry in the care gap work queue presents the patient identifier, the prior result or pattern that triggered the surveillance flag, the time elapsed since the flag-triggering event, the applicable clinical guideline recommendation, and a pre-drafted outreach action comprising a suggested patient communication and a suggested clinical order or referral;
e) accept a single confirmation interaction from a reviewing clinician or care coordinator as a complete outreach action record for each care gap work queue entry when the pre-drafted outreach action is confirmed without modification; and
f) store all outreach action records in an append-only audit registry with the information state at the time of the outreach decision.

38. The system of claim 37, wherein the configurable surveillance rules comprise one or more of: a prior follow-up rule that flags patients with a documented prior Tier 2 result for which the required follow-up action was recorded but no follow-up result has been received within the guideline-specified window; a composite pattern rule that flags patients whose longitudinal result sequence satisfies a pattern rule at a lower individual-result magnitude than the Tier 2 classification threshold; and an incidental finding monitoring rule that flags patients with a documented incidental finding for which guideline-specified follow-up has not been ordered within the required window.

39. The system of claim 37, wherein the care gap work queue is presented as a view separate from the active result disposition queue, and wherein entries in the care gap work queue are prioritized by the time elapsed since the flag-triggering event and the clinical significance of the underlying result type.

40. The system of claim 37, wherein the pre-drafted outreach action for each care gap work queue entry includes a patient communication template expressed in a reading-level-appropriate format, pre-addressed to the patient, and ready for review and transmission through a patient communication channel upon clinician confirmation.

41. The system of claim 37, wherein the proactive patient surveillance function identifies patients whose informal clinical status has not been addressed through the normal result delivery workflow, and wherein the care gap work queue constitutes an externalized, systematic, and auditable representation of the clinical follow-up obligations that would otherwise exist only as an informal memory burden on the reviewing clinician.

42. A computer-implemented system for providing physician-assistive clinical decision governance for abnormal diagnostic results, the system comprising:
a) a contextual assembly engine configured to automatically retrieve and assemble, for each classified abnormal result at the time of presentation to a reviewing clinician, the clinical context required for an informed disposition decision, including at least: the patient's longitudinal history for the result type; a quantitative artifact probability estimate; the applicable clinical guideline recommendation; and a consequence summary for delayed or missed diagnosis;
b) a pre-drafted disposition engine configured to generate a complete, pre-populated disposition record from the assembled contextual elements, selectable by the reviewing clinician through a single confirmation interaction without requiring free-text composition;
c) a tiered queue manager configured to organize pending classified results by acuity level and present high-acuity results for individual review and low-acuity results with pre-drafted dispositions for batch confirmation;
d) a proactive surveillance engine configured to surface patients with unresolved longitudinal patterns or overdue follow-up obligations to the reviewing clinician independent of new result arrival; and
e) an audit registry configured to preserve the complete information state at each disposition decision, including the assembled context presented, the pre-drafted record offered, and whether the clinician confirmed, modified, or rejected each pre-drafted element;
f) wherein the system is designed such that guideline-concordant dispositions require fewer clinician interactions than non-documented dismissal of the same result through existing EHR alert interfaces.

43. The system of claim 42, wherein the system is operable to reduce the average clinician time required for guideline-concordant Tier 1 result disposition to fewer than thirty seconds per result, as measured from result presentation to valid disposition record creation.

44. The system of claim 42, wherein the system presents a personal workload dashboard to the reviewing clinician showing pending queue depth, rolling average disposition time by tier, pre-drafted confirmation rate, and follow-up compliance metrics, and wherein access to individual clinician metrics for aggregate comparison purposes is restricted to authorized quality improvement personnel through role-based access controls.

45. A computer-implemented system for routing unresolved abnormal diagnostic results to a patient advocate safety pathway that operates independently of a clinical escalation hierarchy, the system comprising:
a) one or more processors;
b) one or more non-transitory computer-readable media storing instructions that, when executed by the one or more processors, cause the system to:
i. maintain, in a persistent governance data store independent of any electronic
health record system, a governance state for each classified abnormal diagnostic result and a record of whether each level of a configured clinician escalation hierarchy has received and responded to a disposition notification for the result;
ii. determine, upon expiration of the final configured clinician escalation time window without receipt of a valid clinician disposition record, that all configured clinician escalation levels have been exhausted for the result;
c) upon such determination, concurrently initiate a patient safety pathway by: generating a patient notification expressing the abnormal result, its clinical significance, and applicable guideline-recommended follow-up actions in plain language calibrated to a reading level appropriate for patient communication, and delivering the patient notification through a patient communication channel; and generating a care coordination work queue entry assigned to a designated patient advocate role, wherein the care coordination work queue entry presents the result details, the time elapsed since initial classification, the identity and notification timestamp of each clinician escalation level reached without receipt of a valid disposition record, and a pre-drafted patient outreach action;
d) accept a care coordination record from a user acting in the patient advocate role, wherein the care coordination record satisfies a documentation specification distinct from the clinical disposition documentation requirements of the result's assigned tier;
e) write to an append-only audit registry: the complete clinician escalation history for the result including notification delivery timestamps and non-response periods at each escalation level; the patient notification content and delivery timestamp; and the care coordination record; and
f) maintain the result in an ESCALATED governance state throughout and following the patient safety pathway, such that the care coordination record does not satisfy the clinical disposition documentation requirements of the assigned tier and the clinical governance record of unresolved clinician disposition is preserved independently of and concurrently with the patient safety pathway;
g) wherein the patient safety pathway constitutes a redundant safety loop operating in parallel with the clinical escalation hierarchy, such that the patient notification and care coordination actions are initiated without dependence on clinician response at any escalation level.

46. The system of claim 45, wherein the plain-language patient notification is generated from a template library indexed by result type and tier, and wherein the template is personalized with the patient's name, the specific result value and reference range, the applicable clinical guideline recommendation expressed in non-clinical language, and the name and contact information of the patient's primary care provider.

47. The system of claim 45, wherein the care coordination work queue entry includes a suggested patient communication pre-addressed to the patient and ready for transmission upon patient advocate confirmation, and a suggested clinical order or referral derived from the applicable guideline recommendation, pre-populated for patient advocate review and forwarding to the appropriate clinical service.
